(19) United States
(12) Patent Application Publication
(10) Pub. No.: US 2009/0270628 A1
(43) Pub. Date: Oct. 29, 2009

(54) PROCESS FOR MAKING MONTELUKAST INTERMEDIATES

(76) Inventors: Lambertus Thijs, Wijchen (NL); Henar Tejedor Vinent, Nijmegen (NL)

Correspondence Address: SYNTHON IP INC, 7130 HERITAGE VILLAGE PLAZA, STE 202, GAINESVILLE, VA, 20155, US

(21) Appl. No.: 12/429,652

(22) Filed: Apr. 24, 2009

Related U.S. Application Data
(60) Provisional application No. 61/047,992, filed on Apr. 25, 2008.

(51) Int. Cl. C07D 215/18

(52) U.S. Cl. 546/174

(57) ABSTRACT

A process for making a montelukast intermediate of formula (1) is achieved by reacting a compound of formula (2):

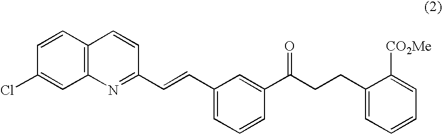

with a hydrogen source in the presence of a ruthenium catalyst of formula (9):

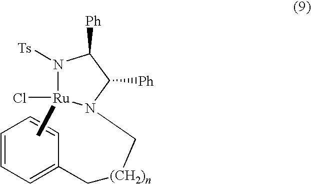

or a dimer thereof, wherein n represents a number from 1 to 3, to form a compound of formula (1):

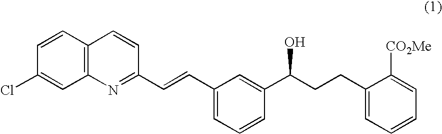

generally in high enantiomeric purity.

### CLAIM TO PRIORITY

Benefit of the following Provisional Applications are claimed under 35 USC § 119(e):

| Application No. | Filing Date |
|---|---|
| 61/047,992 | Apr. 25, 2008 |

This application claims the benefit of priority under 35 U.S.C. § 119(e) from U.S. Provisional application Ser. No. 61/047,992, filed Apr. 25, 2008; the entire contents of which are incorporated herein by reference.

### BACKGROUND OF THE INVENTION

The present invention deals with a process for making the compound methyl [S-(E)]-2-[3-[3-[2-(7-chloro-2-quinolinyl)ethenyl]phenyl]-3-hydroxypropyl]benzoate, which can be represented by the formula (1).

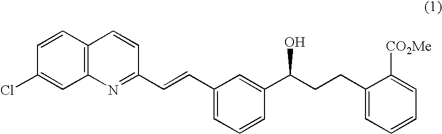

This compound is useful in the synthesis of the pharmaceutical active agent known as montelukast, which is chemically [R-(E)]-1-[[[1-[3-[2-(7-chloro-2-quinolinyl)ethenyl]phenyl]-3-[2-(1-hydroxy-1-methylethyl)phenyl]-propyl]thio]methyl]cyclopropanacetic acid. Montelukast is a leukotriene antagonist, which is effective in the treatment of asthma, allergies, and other associated diseases or conditions.

A known process for making the compound of formula (1) is based on an enantioselective (asymmetric) reduction of the C═O group in the compound of formula (2).

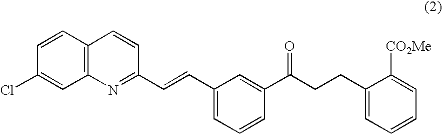

For example, EP 480,717 discloses a process for the preparation of the compound (1) by an asymmetric reduction of (2) using either an oxazaborolidine complex (3) or (−)-B-chlorodiisopinocamphenylborane (4).

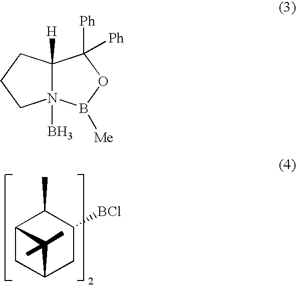

The disadvantage of these reduction agents is that the double bond in the compound of formula (2) may be attacked as well, thus yielding an “overreduced” byproduct, which decreases the yield and can only be removed by relatively complicated procedures. Also, this kind of reduction agent must be used in at least stoichiometric amounts.

U.S. Pat. No. 6,184,381 describes a process for the preparation of optically active secondary alcohols using asymmetric transfer hydrogenation (ATH). One of the process schemes uses, as a hydrogenation catalyst, a transition metal complex, preferably ruthenium complex, modified with an arene and a chiral diamine, in presence of a hydrogen donor. As shown in Table 5, this process was applied to a compound of formula (2) resulting in the reversed (R) enantiomer analogue of formula (1). The ruthenium complex used had the following formula:

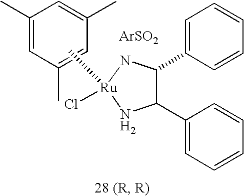

and formic acid was used as the hydrogen donor. The reaction proceeded at room temperature for 72 hours resulting in 68% conversion and the (R) product of 92% ee. Presumably the (S,S) form this ruthenium catalyst would have resulted in the compound of formula (1). A similar disclosure is set forth in the article of Fujii et al. (JACS 1996, 118, 2521-2522), where the ruthenium complex is depicted as

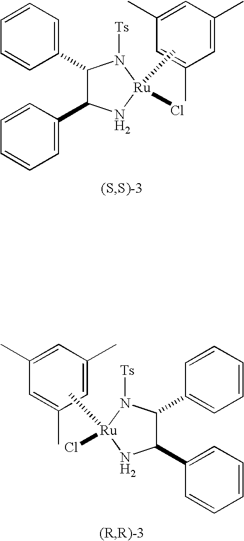

and the (R,R) form is reported to produce the same 68% conversion with 92% ee of the (R) alcohol product (see Table 1) as in U.S. Pat. No. 6,184,381. Fujii et al. further report that this catalyst does not attack the double bond, the chlorine atom, or the ester group in the compound (2). The donor of hydrogen in this hydrogenation process is again formic acid, with an azeotropic mixture of formic acid with triethylamine (in a ratio 2:5) being indicated as generally advantages for such ATH reactions.

WO2006/008562 discloses the use of ATH, using different catalysts than in Fujii et al., to produce the compound of formula (1). The transition metal catalysts in this PCT publication are ruthenium or rhodium complexes containing a sulfamoyl-diamine ligand.

Yet additional catalysts are proposed in US2006/0223999 for producing the compound (1) via ATH. This published U.S. application discloses the use of (R)-methyloxazaborolidine (MeCBS) or the following specific ruthenium catalysts:
trans-RuH(η1BH4)[(R)-2,2′-bis(di-4-tolylphosphino)-1,1′-binaphthyl][(R,R)-1,2-diphenyl-ethylenediamine];
trans-RuCl₂[(R)-2,2′-bis(di-3,5-dimethylphenylphosphino)]-1,1′-binaphthyl][(R,R)-1,2-diphenylethylenediamine]; or
[[N(S),N′(S),1R,2R]-N,N′-bis-[[2-(diphenylphosphino)phenyl]methyl]1,2-cyclo-hexanediamine-N,N′,P,P′]-dichloro-ruthenium.

A variety of transition metal catalysts especially rhodium and rhuthenium based complexes have been applied to ATH reactions in general in order to stereoselectively reduce a carbonyl group to an alcohol group. For example, Hayes et al. discloses a class of ruthenium (II) catalysts including the following tethered complex:

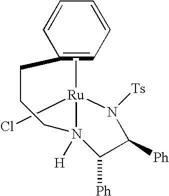

Hayes et al. only uses simple carbonyl compounds as substrates for ATH and does not show the compound of formula (2) as a substrate nor make a compound of formula (1). Similarly, the above complex and many others have been applied to a variety of carbonyl substrates as shown in the poster of Professor Willis and described in his talk at the FAST conference, 2007, University of Cambridge, UK, organized by Johnson Matthey. None of the substrates in the Willis poster correspond to the compound of formula (2) nor do the reductions produce the compound of formula (1).

While a variety of methods for the conversion of the compound (2) into compound (1) have been proposed, an alternative and/or more convenient process is desirable.

### SUMMARY OF THE INVENTION

The present invention relates to a process for producing the montelukast intermediate of formula (1) and hence, more generally, an advantageous process for making montelukast. A first aspect of the invention relates to a process that comprises reacting a compound of formula (2):

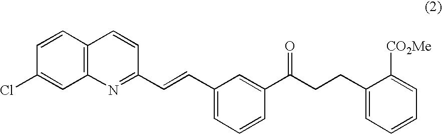

with a hydrogen source in the presence of a chiral ruthenium catalyst of formula (9):

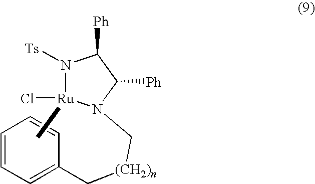

or a dimer thereof, wherein n represents a number from 1 to 3, to form a compound of formula (1):

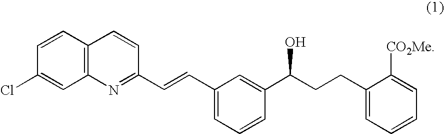

The chiral ruthenium catalyst is typically a complex of the formula (10) or (11)

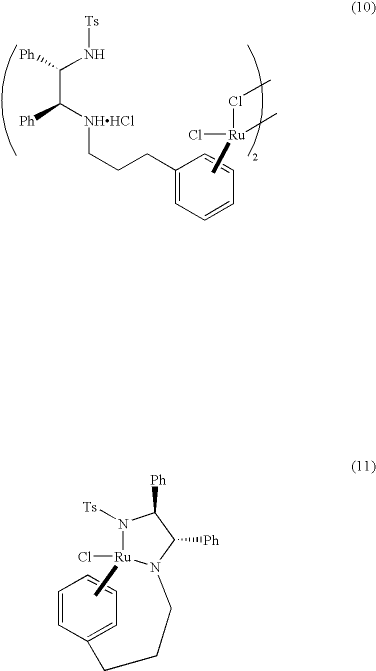

or mixtures thereof. Alternatively, the chiral catalyst is the complex of formula (12) or (13)

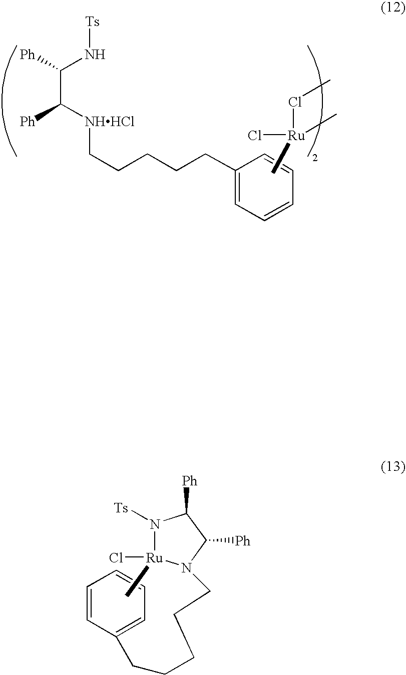

or mixtures thereof. The hydrogen source is typically formic acid and advantageously as an azeotropic mixture with triethylamine.

Another aspect of the invention relates to converting the compound of formula (1) made by the above process into montelukast or a pharmaceutically acceptable salt thereof.

### DETAILED DESCRIPTION OF THE INVENTION

The present invention deals with a process of making the compound of formula (1) by an asymmetric transfer hydrogenation of the compound of formula (2). In the “asymmetric transfer hydrogenation,” the C═O double bond of a substrate is hydrogenated to form an CH—OH bond, wherein the hydrogen atoms are transferred to the substrate from a suitable hydrogen source by the aid of a chiral catalyst, whereby the C-bonded hydrogen is attached asymmetrically, i.e. not in a random, but rather in a rigid, conformation. In this way, an enantio-selective reduction is achieved.

The catalyst used in U.S. Pat. No. 6,184,381 and in Fujii et al. appears to be very selective for the hydrogenation of compound (2); albeit exemplified in the opposite stereoconfiguration resulting in the (R) product instead of the desired (S) orientation of the present invention. Nevertheless, the reaction required a long reaction time and limited temperatures.

It has now been discovered that the use of a catalyst of the formula (9) can provide a more suitable process for the asymmetric transfer hydrogenation reaction of compound (2) into compound (1). The use of a catalyst of formula (9) can provide a shorter reaction time, allows for similar or greater selectivity, and allows for the use of higher temperatures.

In general the process of the present invention comprises reacting a compound of formula (2) (sometimes referred to herein as simply “compound (2)”) with a hydrogen source in the presence of a catalyst of formula (9) to form the compound of formula (1) (sometimes referred to herein as simply “compound (1)”). The compound of formula (2) is known per se and may be obtained from commercial sources or may be made by known process, e.g., by a process disclosed in King et al., J. Org. Chem. 1993, 58, 3731-3735.

The hydrogen source is any compound capable of donating a hydrogen including H₂ or an alcohol such as isopropanol. Preferably, however, the hydrogen source is formic acid. For clarity, the hydrogen source “reacts” with the compound of formula (2) so long as a hydrogen is donated, either directly or indirectly. Thus the hydrogen source “reacts” with the compound of formula (2) even if the hydrogen is first removed from the hydrogen source, such as by thermal action, etc., and then the removed hydrogen bonded to the compound of formula (2). In general the hydrogen source is not elemental hydrogen due to industrial scale handling concerns and/or low reaction rates/conversions and is not an alcohol such as isopropanol due to slightly lower enantiomeric selectivity in the final product. Formic acid is thus the preferred hydrogen source. Formic acid can be used per se but often is provided as a combination of formic acid and a base, e.g., in reagent. Examples of such reagents include formic acid-triethylamine, formic acid-diisopropylethylamine, formic acid-Group (I) or (II) metal bicarbonate, and formic acid-Group (I) or (II) metal carbonate. Generally the most useful combination is a formic acid-triethylamine azeotropic mixture; e.g., about 5:2.

The hydrogen source and/or its reagent form can serve as a solvent for the hydrogenation process. In addition, an inert co-solvent may be added to the system to enhance or completely provide the solvent power. A suitable inert co-solvent includes a liquid hydrocarbon, chlorinated hydrocarbon, an ether including a cyclic ether, a nitrile, and an ester. A typical co-solvent is tetrahydrofuran.

The concentration of the compound (2) in the solvent system is advantageously from 10 to 50 weight percent. In an advantageous mode, both the substrate and the catalyst should be fully dissolved.

The chiral ruthenium catalyst used in the process of the present invention is a complex of formula (9) or a dimer thereof.

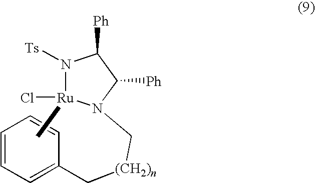

As is understood by workers skilled in the art, “Ph” represents a phenyl group and “Ts” represents a tosylate group. The hydrocarbon linkage between the phenyl ring and the ring nitrogen atom can have 3 to 5 carbon atoms, meaning that “n” has a value of 1-3. The dimer form of a compound of formula (9) refers to the linking together of two structures in ring open form (the Ru—N bonds being extinguished) assumedly via chloro bridging. Additional chloride atoms and/or HCl acid salts are also included within the meaning of a “dimer.” As described below and as set forth in Hayes et al., the dimer form is reached first in the synthesis of the monomer. Typically the compounds are represented by one or more of a complex of formula (10)-(13).

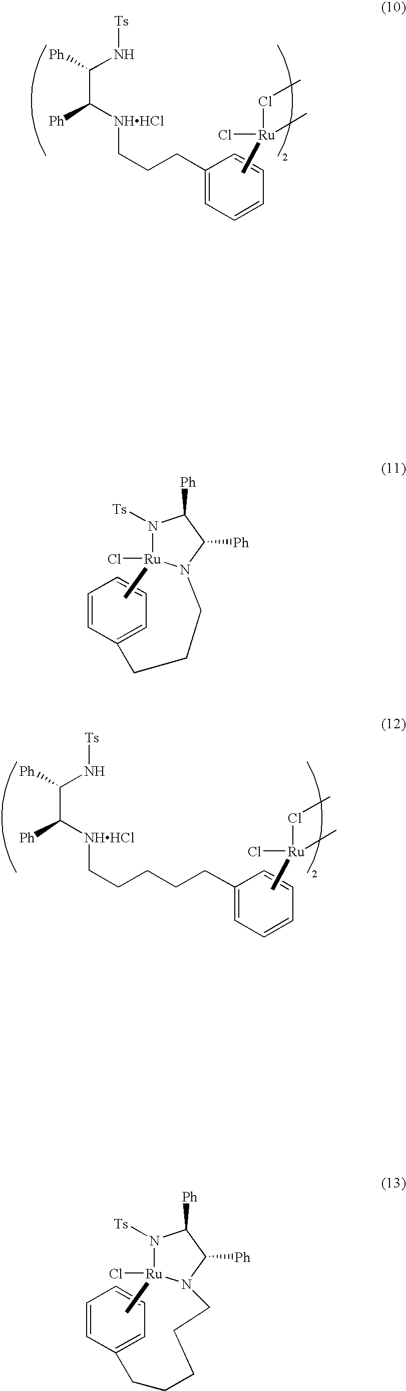

The complex of formula (10) is a representation of a dimer of formula (11), while the complex of formula (12) is a representation of a dimer of formula (13). The dimers generally convert into the more stable monomers. Heating the dimer in the presence of a base (e.g., triethylamine in isopropanol) effectively converts the dimer to the corresponding monomer. Thus the dimer (10) may be used as the catalyst, or the compound may be first converted into the compound (11) and such isolated monomer may be used as the catalyst. Alternatively the compound (10) may be pre-treated by the hydrogen source to convert a part of the compound (10) into compound (11) and the so formed mixture of compounds (10) and (11) may be used as the catalyst. Because the conversion from dimer to monomer happens with relative ease, the dimer will generally be at least partially, if not fully, converted in situ to the monomer under the conditions of ATH. Such an in situ conversion can be arranged by a pretreatment of the dimer with the hydrogen source and/or base in the absence of the carbonyl substrate (2), or, the ATH reaction can proceed directly with conversion of dimer to monomer occurring during the ATH reaction. In the later case, the initial reaction rate may be slow and/or the ATH reaction time may be increased. Thus the dimer, monomer, or combinations thereof can be present while the compound (2) reacts with the hydrogen source. In general the monomer (11) and/or its dimer, which are identified and described in Hayes et al. as (S,S)-3 and its dimer (S,S)-6, are the preferred catalyst(s) for use in the present invention.

In one embodiment, the compound (10) (or other dimer) is treated with the above defined hydrogen source and/or base optionally in an above defined inert co-solvent for a certain time, e.g., 0.5-4 hours, and then the substrate compound (2) is added and the hydrogenation reaction is allowed to proceed. In this embodiment, the dimer (10) is generally substantially converted to the monomer (11) by the time the carbonyl substrate compound (2) is added.

The complexes of formula (9) and the dimers thereof are preparable by general methods known in the art. In particular, the synthetic scheme, and ones analogous thereto, as shown in Hayes et al., JACS 2005, 127, is suitable. By these methods, the ligand is first formed and then RuCl₃, typically as a hydrate, is reacted therewith resulting in the formation of the dimer. As mentioned above, the dimer can be converted to the monomer and isolated for use as a catalyst, or the dimer can be used directly as the catalyst with or without pre-treatment to convert some or all of the dimer into monomer.

To obtain the compound (1) in the desired (S) conformation, the chiral ruthenium catalyst (10) and/or (11) must be present also in a rigid conformation. Thus, for making the compound (1) as the (S)-enantiomer, the catalyst should be made in a (S,S) conformation of the phenyl groups. Conversely the same catalyst with a (R,R) conformation of the phenyl groups shall provide the opposite (R) enantiomer analogue of compound (1). The conversion of the compound (10) into compound (11) runs without change or loss of the original conformation. Accordingly, the process of the present invention, although disclosed and exemplified in respect to the highly preferred (S)-enantiomer of formula (1), may be used also for making the corresponding (R)-counterpart of (1), whenever such making is necessary.

The same is, mutatis mutandis, applicable for the use of compound (12) as such compound is convertible into compound (13) and each of them are equally useful, alone or in a combination, for the hydrogenation of the compound (2).

The amount of the chiral ruthenium catalysts of the present invention is preferably from 0.1 to 1 molar percent in respect to the compound (2). More preferably, the amount of the catalyst is about 0.5 molar percent.

The temperature at which the hydrogenation reaction proceeds is advantageously at least 20° C. and generally no higher than 90° C. Typically temperatures greater than room temperature are preferred as faster reaction times are possible, while too high a temperature tends to lead to reduced selectivity. Accordingly temperatures in the range of 20-60° C., including 30 to 50° C. are commonly used. Within these temperature ranges, the catalyst generally does not cause side reactions (e.g., does not generally cause hydrogenation of the double bond, reduction of the ester group, hydrogenolysis of the chlorine atom, etc.) even at the upper temperature limits.

The reaction conditions, particularly the reaction temperature, are advantageously so selected that the reaction time does not exceed 12 hours, more preferably 8 hours or less, and in some embodiments 6 hours or less. Indeed, as shown in the Example below, using a dimer as the charged catalyst, the reaction can be accomplished in less than 5 hours (e.g. about 4 hours). Charging a monomer as the catalyst could reduce the reaction time. It is an advantage of the catalyst of the present invention that it is possible to convert substantially all the substrate of the formula (2) into the desired compound of formula (1) within such short time periods. The conversion rate may be monitored by a suitable analytical technique, e.g. by HPLC and the reaction may be terminated in proper time.

The reaction typically proceeds with higher than 90% optical selectivity, i.e. the enantiomeric enrichment by the desired enantiomer is higher than 90%, preferably at least 95% ee, and in some embodiments at least 97% ee.

The product (1) can be isolated from the reaction mixture, after the removal of the catalyst, by conventional procedures, e.g., by evaporation of volatile parts of the reaction mixture, redissolution in a second solvent (with an optional purification of the solution, e.g. by an adsorbent or by an extraction) and precipitation from the second solvent. The precipitated product (1) may be further purified by a recrystallization or by a chromatography, if desired.

The formed compound (1) may be easily obtained in a higher than 95% chemical purity and in higher than 95% enantiomeric enrichment. Such a quality is suitable for use in further applications, e.g. for the synthesis of montelukast and/or its pharmaceutically acceptable salts. The conversion of the compound of formula (1) to montelukast and its salts, especially sodium montelukast is well known.

The invention is illustrated by the following example.

### Example

### Synthesis of Methyl [S-(E)]-2-[3-[3-[2-(7-chloro-2-quinolinyl)ethenyl]phenyl]-3-hydroxypropyl]benzoate

Substrate:

Methyl 2-(3-(3-(2-(7 Chloro-2-quinolinyl)ethenyl)-phenyl)-3-oxopropyl)benzoate

Ruthenium Catalyst:

N-[(1S,2S)-1,2-diphenyl-2-(3-phenylpropylamino)-ethyl]-4′-methylbenzenesulfonamide dichlororuthenium hydrochloride]dimer (compound 10).

A suspension of 7 mg of the ruthenium catalyst in 1 ml azeotrope Et₃N/Formic acid 5:2 was stirred at 28° C. for 30 minutes up to dissolution. A solution of 911 mg of the substrate in 2 ml THF was added to above solution. (Note: heat was needed in order to dissolve the substrate in the THF). The temperature was increased to 40° C., and the reaction mixture was kept at this temperature for 4 hours 15 min. Reaction progress was monitored by HPLC. The solution obtained was allowed to reach the ambient temperature. It was filtered over cellite, washed with 100 ml (EtOAc/heptane 1:1) and concentrated to a reddish liquid. The liquid was dissolved in 8 ml MeOH. To this solution 0.8 ml water was added slowly. The formed suspension was stirred overnight at ambient temperature. Then it was filtered and dried at air.

The product can be recrystallized from 8 ml MeOH/0.8 ml H₂O or 6 ml MeOH/0.6 ml H₂O.

The ee purity is 97.23%

HPLC purity is 96.8%

Each of the patents, patent applications, and journal articles mentioned above are incorporated herein by reference. The invention having been described it will be obvious that the same may be varied in many ways and all such modifications are contemplated as being within the scope of the invention as defined by the following claims.

We claim:

1. A process which comprises reacting a compound of formula (2): 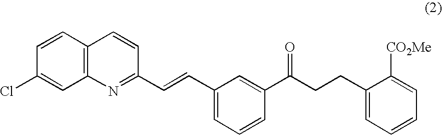
with a hydrogen source in the presence of a ruthenium catalyst of formula (9): 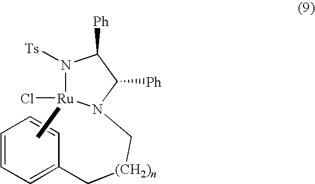
or a dimer thereof, wherein n represents a number from 1 to 3, to form a compound of formula (1):

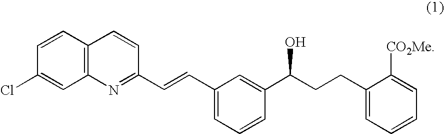

2. The process according to claim 1, wherein said hydrogen source is formic acid.

3. The process according to claim 2, wherein said formic acid is present as a combination of formic acid and triethylamine.

4. The process according to claim 2, wherein said ruthenium catalyst is a complex of formula (10) or (11):

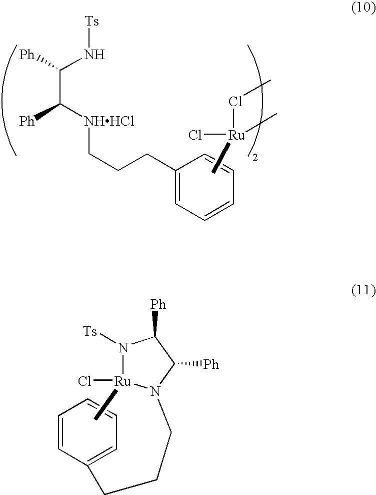

5. The process according to claim 4, wherein said reaction is carried out at a temperature within the range of 20 to 60° C.

6. The process according to claim 5, wherein said reaction is carried out at a temperature within the range of 30 to 50° C.

7. The process according to claim 5, wherein said reaction is carried out for less than 12 hours.

8. The process according to claim 7, wherein said compound of formula (1) is produced in enantiomeric purity of at least 97%.

9. The process according to claim 5, wherein said reaction is carried out for less than 8 hours.

10. The process according to claim 9, wherein said compound of formula (1) is produced in enantiomeric purity of at least 95%.

11. The process according to claim 6, wherein said reaction is carried out for less than 8 hours and said compound of formula (1) is produced in enantiomeric purity of at least 95%.

12. The process according to claim 11, wherein said compound of formula (1) is produced in enantiomeric purity of at least 97%.

13. The process according to claim 1, which further comprises converting said compound of formula (1) into montelukast or a pharmaceutically acceptable salt thereof.

14. The process according to claim 4, which further comprises converting said compound of formula (1) into montelukast or a pharmaceutically acceptable salt thereof.

15. The process according to claim 6, which further comprises converting said compound of formula (1) into montelukast or a pharmaceutically acceptable salt thereof.

16. The process according to claim 8, which further comprises converting said compound of formula (1) into montelukast or a pharmaceutically acceptable salt thereof.

17. The process according to claim 9, which further comprises converting said compound of formula (1) into montelukast or a pharmaceutically acceptable salt thereof.

18. The process according to claim 10, which further comprises converting said compound of formula (1) into montelukast or a pharmaceutically acceptable salt thereof.

19. The process according to claim 11, which further comprises converting said compound of formula (1) into montelukast or a pharmaceutically acceptable salt thereof.

20. The process according to claim 12, which further comprises converting said compound of formula (1) into montelukast or a pharmaceutically acceptable salt thereof.
